(19) United States
(12) Patent Application Publication
(10) Pub. No.: US 2009/0196924 A1
(43) Pub. Date: Aug. 6, 2009

(54) CONTROLLED-RELEASE LAMOTRIGINE FORMULATIONS

(76) Inventors: Pramod Kharwade, Chhindwara (IN); Narayanan Badri Vishwanathan, Chennai (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/364,708

(22) Filed: Feb. 3, 2009

Related U.S. Application Data
(60) Provisional application No. 61/025,878, filed on Feb. 4, 2008.

(51) Int. Cl. A61K 9/26; A61K 31/53; A61P 25/08

(52) U.S. Cl. 424/469; 514/242

(57) ABSTRACT

Pharmaceutical controlled-release formulations comprising particles comprising lamotrigine or a pharmaceutically acceptable salt thereof, coated with a modified-release coating comprising a modified-release coating agent and a pore-former.

The present invention relates to pharmaceutical controlled- or modified-release formulations comprising lamotrigine or its pharmaceutically acceptable salts. More particularly, the present invention relates to controlled-release pharmaceutical formulations providing prolonged release of lamotrigine from the dosage form. The invention also relates to processes of preparation of pharmaceutical formulations and method of using formulations for treating epilepsy.

Tablet products containing lamotrigine are available globally from GlaxoSmithKline with the brand names LAMICTAL™ and LAMICTAL™ CD. GlaxoSmithKline is also developing an extended-release product for once-daily dosing containing lamotrigine, to be sold under the brand name LAMICTAL™ XR.

Lamotrigine has a chemical name 3,5-diamino-6-(2,3-dichlorophenyl)-as-triazine, its molecular formula is C₉H₇N₅C₁₂, and its molecular weight is 256.09. Lamotrigine is a white to pale cream-colored powder and has a pKₐ of 5.7. Lamotrigine is very slightly soluble in water (0.17 mg/mL at 25° C.) and slightly soluble in 0.1 M HCl (4.1 mg/mL at 25° C.). The structural formula is shown below.

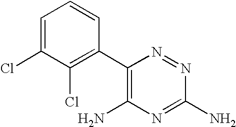

Lamotrigine is prescribed for conversion to monotherapy in adults with partial seizures who are receiving treatment with carbamazepine, phenyloin, phenobarbital, primidone, or valproate as the single antiepileptic drug.

Lamotrigine is also prescribed as adjunctive therapy for partial seizures, the generalized seizures of Lennox-Gastaut syndrome, and primary generalized tonic-clonic seizures in adults and pediatric patients (at least two years of age).

Lamotrigine is indicated for the maintenance treatment of Bipolar I Disorder to delay the time to occurrence of mood episodes (depression, mania, hypomania, mixed episodes) in patients treated for acute mood episodes with standard therapy.

The precise mechanisms by which lamotrigine exerts its anticonvulsant action are unknown. One proposed mechanism of action of lamotrigine, the relevance of which remains to be established in humans, involves an effect on sodium channels. In vitro pharmacological studies suggest that lamotrigine inhibits voltage-sensitive sodium channels, thereby stabilizing neuronal membranes and consequently modulating presynaptic transmitter release of excitatory amino acids (e.g., glutamate and aspartate).

Lamotrigine has been used to treat over a million patients worldwide, including about 4000 adults and over 1000 children in clinical trials, Extensive experience with lamotrigine has indicated that it may be effective when other anticonvulsant drugs have failed. It is a valuable broad-spectrum drug that is well tolerated and has few adverse effects apart from skin rash.

Adverse events associated with lamotrigine are typical of antiepileptic drugs, namely dizziness, ataxia, diplopia, somnolence, headache, and asthenia.

Neurological side effects are normally seen at higher plasma concentrations (which are most likely to occur at peak plasma concentrations).

Dose reduction and slow dosage escalation are two techniques to overcome these peak time side effects.

The present invention will reduce these side effects by controlling the Cₘₐₓ of lamotrigine by the use of a controlled release formulation of lamotrigine. It will also maintain the steady state concentration with little fluctuations. The reduced incidence of these neurological side effects will improve patient compliance with the therapy.

Serious skin reactions (including Stevens-Johnson syndrome and toxic epidermal necrolysis) occurring in patients taking lamotrigine was reported. Skin reactions such as Stevens-Johnson syndrome are potentially fatal and have an incidence of 1 in 1000 person-years in adults. The incidence is higher in children. Risk factors for skin reactions include high plasma concentration, concomitant sodium valproate therapy (Valproate reduces the hepatic clearance of lamotrigine thereby increasing plasma concentrations of the drug by approximately two-fold for a given dose), a high initial dose of lamotrigine and rapid dose escalation.

There is reported data that suggests slow dosage escalation or titration when initiating therapy may lessen the likelihood of development of severe rash.

Controlled-release lamotrigine, which is designed to avoid excessive Cₘₐₓ levels, will produce lower plasma concentrations, which are reached over a longer period of time and will reduce the incidence of this troublesome side effect of lamotrigine. Further, the controlled release formulation will be much safer to use with concomitantly administered drugs such as phenyloin, carbamazepine, sodium valproate etc.

Presently, lamotrigine is prescribed in conventional tablets or dispersible/chewable tablet form in doses ranging from 25 to 600 mg/day, once or in two divided doses daily. Immediate release dosage forms provide rapid dissolution results with a rapid increase in blood plasma levels after each dosing, which causes adverse effects. The reason for giving divided doses of lamotrigine is to prevent very high concentrations in the plasma, which can occur with single daily doses of the conventional formulation.

U.S. Patent Application Publication No. 2004/0043996 discloses a multi-particulate controlled release dosage formulation of lamotrigine or a pharmaceutically acceptable salt thereof, which comprises: (a) particles comprising lamotrigine; (b) a release rate-controlling polymer; and (c) a rapidly disintegrating binder, which will allow the particles to rapidly disperse in an aqueous environment.

U.S. Patent Application Publication No. 2005/0238724 discloses a plurality of lamotrigine particles having a specific surface area of from about 2 to about 3.5 square meters per gram.

U.S. Patent Application Publication No. 2004/0192690 discloses a sustained release formulation of lamotrigine or a pharmaceutically acceptable derivative thereof and methods of treatment and uses.

U.S. Pat. No. 5,004,614 discloses a device for controlled release of an active agent, comprising a core comprising an active agent and a release modifying agent and an outer coating covering said core, the thickness of said coating being adapted such that it is substantially impermeable to the entrance of an environmental fluid present in an environment of use and substantially impermeable to the exit of said active agent during a dispensing period, said coating including an orifice extending substantially completely through said coating but not penetrating said core and communicating from said environment of use to said core for allowing the release of said active agent into said environment of use, said orifice having an area from about 10 to about 60 percent of the face area of said device, the rate limiting step for the release of said active agent substantially being the exit of said active agent through said orifice via one or more of dissolution, diffusion or erosion of said active agent in solution or suspension, said release modifying agent enhancing or hindering the release of said active agent depending upon the solubility and/or effective solubility of said active agent in said environment of use.

The present invention can reduce these side effects by controlling the plasma levels of lamotrigine through the use of a controlled-release formulation of lamotrigine. It will also maintain the steady state concentration with little fluctuations. The reduced incidence of these neurological side effects improves patient compliance with their prescribed therapy.

### SUMMARY

An aspect of the present invention provides low plasma concentrations of lamotrigine with controlled-release dosage forms.

An aspect of the present invention provides once daily dosage forms of lamotrigine that release lamotrigine into the blood stream over a prolonged period of time.

An aspect of the present invention provides matrix tablets using hydrophilic substances, optionally an enteric coating, and optionally the enteric coating comprising a pore-former.

An aspect of the present invention provides matrix tablets using hydrophobic substances, optionally an enteric coating, and optionally the enteric coating comprising a pore-former.

An aspect of the present invention includes modifying the surface of lamotrigine with surface modifying agents and compression into tablets, optionally an enteric coating, and optionally the enteric coating comprising a pore-former.

An aspect of the present invention provides matrix tablets using acrylic polymers, optionally an enteric coating, and optionally the enteric coating comprising a pore-former.

An aspect of the present invention provides immediate release tablets coated with one or more of rate-controlling polymers and pH-dependent polymers.

An aspect of the present invention provides multi-particulate controlled-release dosage forms comprising lamotrigine.

An aspect of the present invention provides delayed-release and extended-release multi-particulates comprising lamotrigine, which can be combined and filled into capsules or compressed into tablets.

### DETAILED DESCRIPTION

The present invention relates to pharmaceutical controlled-release formulations comprising lamotrigine or its pharmaceutically acceptable salts. More particularly, the present invention relates to controlled-release pharmaceutical formulations providing prolonged release of lamotrigine from the dosage forms. The invention also relates to processes of preparation of formulations and methods of using the formulations for treating epilepsy.

The present invention also relates to development of solid controlled-release oral dosage forms comprising lamotrigine.

The present invention further relates to matrix tablets comprising lamotrigine with hydrophilic substances, optionally coating the matrix tablets with enteric coating polymers, and optionally the enteric coating containing a pore-former.

As used herein the term “lamotrigine” includes the compound lamotrigine, prodrugs thereof, active metabolites of lamotrigine, prodrugs thereof, and any of their polymorphs, solvates and hydrates.

The term “pharmaceutically acceptable salt” refers to salts of lamotrigine and active metabolites of lamotrigine, and said salts may be prepared using pharmaceutically acceptable acids. Suitable pharmaceutically acceptable salts include but are not limited to the chloride and other halogen salts, and salts such as are formed by reaction of lamotrigine with acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pathothenic, phosphoric, p-toluenesulfonic, succinic, sulfuric, and tartaric acids, and the like.

According to the present invention, lamotrigine and its salts can be used in any crystalline form, or in amorphous form, or in any combinations thereof.

As used herein the term “controlled- or modified-release” means the release of the active substance, e.g., lamotrigine chloride, from a pharmaceutical dosage form in a manner modified to occur at a different time and/or at a different rate than that obtained from an immediate release product, such as a conventional swallowed tablet or capsule. Sometimes the active substance may be present in “sustained-release” form where the release of the active substance is modified to occur over a prolonged period of time. Sometimes the active substance may be present in “delayed-release” form where the release of the active substance is modified to commence at a later time than that from an immediate release form. Controlled-release formulations can exhibit sustained-release characteristics, delayed-release characteristics, or a combination thereof.

Controlled-release pharmaceutical formulations of the present invention release drug over periods of time at least about 6 hours, or at least about 8 hours, or at least about 12 hours, or at least about 16 hours, or at least about 20 hours, following administration.

The terms “release controlling polymers or agents,” or “release modifying polymers or agents” in the present context means any polymer or agent capable of increasing or retarding in vitro and/or in vivo drug release from the composition.

Lamotrigine is very slightly soluble in water (0.17 mg/mL at 25° C.). Since lamotrigine is a slightly soluble drug, particle size, particle size distribution, and surface area become important for pharmaceutical compositions and formulations, particularly when controlled release compositions are desired. For an insoluble drug, particle size can play an important role in enhancing the solubility of the drug.

Particle size reduction increases the surface area of the solid phase that is in contact with a liquid medium. The particle size distributions according to the present invention provide an enhanced rate of dissolution of the lamotrigine.

Particle size distributions are commonly expressed in terms of, D₁₀, D₅₀, D₉₀ and D_{[4,3]}. The D₁₀, D₅₀ and D₉₀, also represented as D(0.1), D(0.5) and D(0.9), values represent the 10th, the median or 50th percentile, and the 90th percentile of the particle size distribution, respectively, as measured by volume. That is, the D₁₀, D₅₀, and D₉₀ are values of the distribution such that 10%, 50%, and 90% of the particles have a volume percentage of the given value or less, or is the percentage of particles smaller than that size. D_{[4,3]} is the volume weighted mean or mass moment mean diameter of the particles, or the volume weighted particle size. D_{[3,2]} is the surface weighted mean, also known as the surface area moment mean diameter or Sauter mean diameter. Other parameters showing the particle size distribution include uniformity, span value, specific surface area. Particle size distributions frequently are measured using a laser light diffraction instrument such as a Malvern particle size analyzer (Malvern Instruments Ltd., Malvern, Worcestershire, United Kingdom).

In an embodiment the invention relates to pharmaceutical compositions comprising lamotrigine of the particle size distribution wherein D₁₀ is less than about 100 μm, D₅₀ is less than about 200 μm, and D₉₀ is less than about 400 μm.

In an embodiment of the present invention, a tablet formulation contains lamotrigine, hydroxypropyl methylcellulose, and other additives such as diluents and lubricants, the hydroxypropyl methylcellulose present being in the formulation to determine the in vitro and in vivo release characteristics of the composition.

The present invention further relates to matrix tablets comprising lamotrigine with hydrophobic substances, optionally coating the matrix tablets with an enteric coating polymer, and optionally the enteric coating containing a pore-former.

The present invention further relates to immediate-release tablets coated with a single release controlling polymer, a pH-dependent polymer, or a mixture thereof.

In an embodiment of the present invention, tablet formulations of lamotrigine comprise one or more hydrophobic polymers such as ethylcellulose, methacrylic polymers, etc., and other additives such as diluents and lubricants, where the hydrophobic polymer present in the formulation will determine the in vitro and in vivo drug release characteristics of the formulation.

The in vitro and in vivo release characteristics of a tablet formulation can be tailored for specific requirements; this can be achieved by any of several established techniques or methods such as that described above.

Another method includes altering the drug release characteristics by coating, a release rate-modifying coating being employed to retard or delay the release rate.

A release rate-modifying coating can be applied to immediate-release or modified-release core compositions. To further facilitate drug release from the core, channels or pores can be incorporated in the coating.

Channels or pores can be formed in situ in a coating when the coating layer is principally made up of water-insoluble polymer, or the channels as described herein can be made mechanically during dosage form manufacturing, for example using mechanical drills or laser drilling techniques.

In situ pore forming can be achieved by adding a water-soluble component into a water-insoluble or pH-dependent soluble coating layer.

Pore-forming excipients, if they are soluble in all pH conditions, will be dissolved once a tablet is present in an aqueous fluid, thereby forming irregular pores on the surface of the tablet. For example, the pores or channels created in a water-insoluble coating when the tablet comes in contact with aqueous fluids will facilitate drug leaching from the core, and can facilitate disintegration. Similarly, for tablet formulations with pH-dependent coatings, the tablet remains intact in low pH conditions, while the pore-forming excipient would dissolve and thus can modulate a drug release under those conditions.

In embodiments, the pores might not sufficiently extend through the thickness of the coating to reach the core, but will form channels or networks of pores that can interconnect from the outer surface of the coating to the outer surface of the core.

In an embodiment, a core tablet comprising lamotrigine is prepared by mixing lamotrigine with immediate-release excipients and compressing the mixture into tablets, then this core tablet is coated with one or more rate-controlling polymers, or one or more semi-permeable polymers, or mixtures thereof.

In an embodiment, tablet formulations of the present invention comprise an immediate-release tablet which is coated with a release-modifying polymer, the release-modifying polymer having pH-independent solubility.

In an embodiment, tablet formulations of the present invention comprise a modified-release tablet which is coated with a release-modifying polymer, the release-modifying polymer having pH-independent solubility.

In an embodiment, tablet formulations of the present invention comprise an immediate-release tablet which is coated with a release-modifying polymer, the release polymer having pH-dependent solubility.

In an embodiment, tablet formulations of the present invention comprise a modified-release tablet which is coated with a release-modifying polymer, the release polymer having pH-dependent solubility.

In an embodiment, tablet formulations of the present invention comprise an immediate-release tablet which is coated with a release-modifying polymer, the release polymer being a water-insoluble polymer.

In an embodiment, tablet formulations of the present invention comprise a modified-release tablet which is coated with a release-modifying polymer, the release polymer being a water-insoluble polymer.

In an embodiment, tablet compositions of the present invention comprise an immediate-release tablet which is coated with a release-modifying polymer, the release polymer having pH-independent solubility, and further to facilitate drug release a water-soluble pore former or channelizer is incorporated into the modified-release coating.

In an embodiment, tablet formulations of the present invention comprise a modified-release tablet which is coated with a release-modifying polymer, the release polymer having pH-independent solubility, and further to facilitate drug release a water-soluble pore former or channelizer is incorporated into the modified-release coating.

In an embodiment, tablet formulations of the present invention comprise an immediate-release tablet which is coated with a release-modifying polymer, the release-modifying polymer having pH-dependent solubility, and further to facilitate drug release a water-soluble pore-former or channelizer is incorporated into the modified-release coating.

In an embodiment, tablet formulations of the present invention comprise a modified-release tablet which is coated with a release-modifying polymer, the release polymer having pH-dependent solubility, and further to facilitate drug release a water-soluble pore-former or channelizer is incorporated into the modified-release coating.

In an embodiment, tablet formulations of the present invention comprise an immediate-release tablet which is coated with a release-modifying agent, the rate-modifying agent used being insoluble in water, and further to facilitate drug release a water-soluble pore-former or channelizer is incorporated into the modified-release coating.

In an embodiment, tablet formulations of the present invention comprise a modified-release tablet which is coated with a release-modifying agent, the rate modifying agent being water-insoluble, and further to facilitate drug release a water-soluble pore-former or channelizer is incorporated into the modified-release coating.

In an embodiment, the invention further relates to monolithic, bilayered or multilayered matrix tablets, optionally having the matrix tablets coated with enteric coating polymers, and optionally the enteric coating containing a pore-former.

In an embodiment of the present invention, the surfaces of lamotrigine particles are coated with surface-modifying agents that modify the solubility of lamotrigine, the particles are compressed into tablets, optionally the compressed tablets are coated with enteric coating polymers, and optionally the enteric coating contains a pore-former.

The present invention further relates to delayed-release and extended-release multi-particulates comprising lamotrigine, which can be combined and filled into capsules or compressed into tablets.

It has been found that solubility of lamotrigine is moderately high in all media, and is slightly pH-dependent, with the highest solubility in 0.1N HCl.

In one embodiment, lamotrigine is mixed with surface-modifying excipients and the rate of solubility of lamotrigine is retarded, and the resultant mixture can be filled into capsules or compressed into tablets.

The surface-modifying excipients comprise substances that can retard the solubilization rate of lamotrigine, including but not limited to waxes, stearic acid, magnesium stearate, ethylcellulose, hydrogenated castor oil, glyceryl monostearate, glyceryl behenate, and the like.

In an embodiment, lamotrigine is mixed with hydrophilic or hydrophobic rate-retarding polymers, or mixtures of both, using wet granulation or dry granulation processes, the granules are then mixed with other extragranular excipients and compressed into tablets, and the tablet may be coated with pH-dependent polymers which, when administered into the gastrointestinal tract, release the drug from the pharmaceutical formulations at desired pH conditions, and the pH-dependent coating optionally comprises one or more pore-formers.

Useful hydrophilic excipients that form a matrix with lamotrigine include, but are not limited to, sodium carboxymethylcelluloses, hydroxypropylcelluloses, hydroxyethylcelluloses, hydroxypropyl methylcelluloses, carboxymethylamides, potassium methacrylate/divinylbenzene co-polymers, polymethylmethacrylates, polyvinylpyrrolidones, polyvinylalcohols, methylcelluloses, carboxymethylcelluloses, polyoxyethyleneglycols, xanthan gum, carbomers, poly(ethylene oxide) polymers (Polyox™ from Dow Chemical Co.), hydrocolloids such as natural or synthetic gums, cellulose derivatives in addition to than those listed above, carbohydrate-based substances such as acacia, gum tragacanth, locust bean gum, guar gum, agar, pectin, carrageen, soluble alginates, carboxypolymethylene, and the like.

Hydrophobic excipients that are useful in the compositions include, but are not limited to, ethylcelluloses, magnesium stearate, stearic acid, hydrogenated castor oil, glyceryl monosterate, glyceryl behenate, talc, etc.

Various pore-forming agents useful in the compositions include, but are not limited to: inorganic salts such as sodium chloride and potassium chloride; sugars such as lactose, sucrose, mannitol and sorbitol; hydroxylated compounds, including polyvinyl alcohols and glycols, such as polyethylene glycol and propylene glycol; cellulose derived materials, such hydroxypropyl celluloses, hydroxypropyl methylcelluloses, and hydroxyethyl celluloses; methacrylic acid copolymers; disintegrants such as croscarmellose sodium, crospovidone, and sodium starch glycolate; water soluble polymers such as polyvinylpyrrolidones (povidones or “PVP”); miscellaneous agents such as talc and silicon dioxide; gelling agents such as carbomers and xanthum gum; and mixtures of any two or more thereof.

Various enteric coating polymers useful in the composition include, but are not limited to, cellulose acetate phthalates, hydroxypropyl methylcellulose phthalates, polyvinylacetate phthalates, hydroxypropyl methylcellulose acetate succinates, hydroxypropyl methylcellulose succinates, cellulose acetate trimellitates, cellulose acetate phthalates, cellulose acetate maleates, cellulose acetate butyrates, cellulose acetate propionates, copolymers of methyl methacrylic acid and methacrylate, copolymers of methyl acrylate, methyl methacrylate and methacrylic acid, ethyl methylacrylate-methylmethacrylate-chlorotrimethylammonium ethyl acrylate copolymers, polyvinyl phthalates, natural resins such as zein, shellac, commercially available enteric dispersion systems and any combinations of such materials.

Various commercially available enteric coating materials that can be used in current formulations include, but are not limited to, methacrylic acid-methacrylate copolymer, acid number of 180 to 200 (Eudragit™ S), HPMC E15 LV, hydroxypropyl methylcellulose phthalate (HP™ 55, available from Shinetsu Chemical Co., Tokyo, Japan), cellulose acetate phthalate, ethylcellulose phthalate, hydroxypropyl methylcellulose succinate, cellulose acetate succinate, hydroxypropyl methylcellulose hexahydrophthalate, cellulose acetate hexahydrophthalate, hydroxypropyl methylcellulose trimellitate, methacrylic acid-methacrylate copolymer (acid number 300 to 330, also known as Eudragit™ L). Different grades of useful Eudragit™ products include but are not limited to Eudragit RS 12.5, 100 grades (low permeability), Eudragit RL 12.5, 100 grade (high permeability), Eudragit™ L100-55 (methacrylic acid and ethyl acrylate copolymer) soluble at a pH above 5.5 and practically insoluble at pH below 5.5; Eudragit™ L100 (soluble at pH above 6 and practically insoluble at pH below 6), Eudragit™ S100, soluble at pH above 7 and practically insoluble at pH below 7. Eudragit™ L100 and Eudragit™ S100 are methacrylic acid and methyl methacrylate copolymers, all of the Eudragit™ products being available from Evonik Industries AG, Essen, Germany.

Various semi-permeable coating materials that can be used for the formulations include, but are not limited to, cellulose acetate, cellulose diacetate, cellulose triacetate, cellulose propionate, cellulose acetate butyrate, cellulose trivalerate, cellulose trilaurate, cellulose tripalmitate, cellulose trisuccinate, cellulose triheptylate, cellulose tricaprylate, cellulose trioctanoate, cellulose tripropionate, cellulose dicaprylate and cellulose dipentanate, cellulose acetate valerate, cellulose acetate succinate, cellulose propionate succinate, cellulose acetate octanoate, cellulose valerate palmitate, cellulose acetate palmitate, cellulose acetate heptanoate, cellulose acetate acetoacetate, cellulose acetate chloroacetate, cellulose acetate furoate, dimethoxyethyl cellulose acetate, cellulose acetate carboxymethoxypropionate, cellulose acetate benzoate, cellulose butyrate naphthylate, cellulose acetate benzoate, methylcellulose acetate, methylcyanoethyl cellulose, cellulose acetate methoxyacetate, cellulose acetate ethoxyacetate, cellulose acetate dimethylsulfamate, ethylcellulose, ethylcellulose dimethylsulfamate, cellulose acetate p-toluene sulfonate, cellulose acetate methylsulfonate, cellulose acetate dipropylsulfamate, cellulose acetate butylsulfonate, cellulose acetate laurate, cellulose stearate, cellulose acetate methylcarbamate, agar acetate, amylose triacetate, beta glucan acetate, beta glucan triacetate, acetaldehyde dimethyl acetate, cellulose acetate ethyl carbamate, cellulose acetate phthalate, cellulose acetate dimethyl aminoacetate, cellulose acetate ethyl carbonate, poly(vinylmethyl)ether copolymers, cellulose acetate with acetylated hydroxyethyl cellulose hydroxylated ethylenevinylacetate, poly(orthoester)s, polyacetals, semipermeable polyglycolic, polylactic acid and derivatives thereof, ethyl cellulose, and the like.

Further, the present invention includes processes for preparing formulations containing lamotrigine, wherein an embodiment of a process comprises:

1) sifting active ingredients and excipients through a sieve;

2) dry mixing sifted active ingredients and excipients, or only excipients;

3) optionally, blending the step 2) dry mixture with a lubricant;

4) optionally, compacting and milling the dry mixture of step 2) to form granules;

5) optionally, granulating the dry mixture of step 2) using a granulating solution with or without an active ingredient, drying the wet mass of granules, and sizing dried granules through a sieve;

6) blending granules from step 4) and/or step 5) with desired extragranular excipients;

7) compressing the blend of step 3) or step 6) into tablets, or alternatively filling into capsules; and

8) optionally, coating compressed tablets with film-coating polymers, pH-dependent polymers, rate-controlling polymers, or mixtures thereof.

Alternatively, multi-particulate systems of lamotrigine can be prepared, an embodiment of a process comprising:

1) dissolving or dispersing lamotrigine in a solvent and optionally adding an excipient to the dispersion or solution;

2) spraying the drug solution or dispersion of step 1) onto an inert substrate;

3) optionally coating the product of step 2) with a rate-retarding polymer, pH dependent polymer, or mixture thereof; and

4) mixing the product of step 2) or step 3) with desired excipients and compressing into tablets or filling into capsules.

Drug release characteristics from pharmaceutical dosage forms can be studied, frequently using the procedures described in Test 711 “Dissolution,” United States Pharmacopeia 29, United States Pharmacopeial Convention, Inc., pages 2673-2682, 2005 (“USP”).

Certain specific aspects and embodiments of the invention will be further described by the following examples, which are provided solely for purposes of illustration and are not intended to limit the scope of the invention in any manner.

### EXAMPLE 1

### Matrix Tablets with Hydrophilic Excipients

| Ingredient | mg/Tablet |
|---|---|
| Lamotrigine | 200 |
| Hydroxypropyl methylcellulose | 100 |
| (Methocel ™ K100LVCR) | |
| Lactose | 98 |
| Magnesium stearate | 2 |

Methocel products are supplied by Dow Chemical Co., Midland, Mich. U.S.A.

Manufacturing Process:

1) Lamotrigine, Methocel K100LVCR and lactose were sifted through a BSS #40 sieve and mixed together.

2) Magnesium stearate was sifted through a BSS #60 sieve and mixed with the step 1 blend.

3) The blend was compressed into tablets using 14.7×6.7 mm punches.

In vitro dissolution of the tablets was studied using the USP procedure and the conditions: 900 ml of 0.1 N HCl, USP type 2 apparatus, 50 rpm stirring. Results are in the table below:

| | Cumulative % of |
|---|---|
| Hours | Drug Dissolved |
| 1 | 20 |
| 2 | 35 |
| 4 | 61 |
| 8 | 92 |
| 12 | 101 |

### EXAMPLE 2

### Matrix Tablets with Hydrophilic Excipients, Enteric Coating and a Pore-Former

| | |
|---|---|
| Ingredient | mg/Tablet |

Core Tablet

| | |
|---|---|
| Lamotrigine | 200 |
| Hydroxypropyl methylcellulose | 45.36 |
| (Methocel ™ K4M) | |
| Hydroxypropyl methylcellulose | 62.64 |
| (Methocel ™ K100LVCR) | |
| Lactose | 90.4 |
| Magnesium stearate | 1.6 |

Coating

| | |
|---|---|
| Methacrylic acid-ethyl acrylate | 24.38 |
| polymer (Eudragit ™ L30D55) | |
| Povidone K30 | 7.314 |
| Triethyl citrate | 2.854 |
| Talc | 2.518 |
| Water* | 163 |

*Evaporates during processing.

Eudragit products are supplied by Evonik Industries, Germany.

Manufacturing Process:

Core Tablets:

1) Lamotrigine, Methocel K4M, Methocel K100LVCR and lactose were sifted through a BSS #40 sieve and mixed together.

2) Magnesium stearate was sifted through a BSS #60 sieve and mixed with step 1 blend.

3) The blend was compressed into tablets using 14.7×6.7 mm punches.

Coating:

4) Triethyl citrate, talc and povidone were combined with water and stirred.

5) Eudragit L30D55 was added to the step 4 dispersion and stirred at a slow speed for 45 minutes.

6) Step 3 tablets were coated with step 5 coating dispersion to produce a 5% weight gain.

### EXAMPLE 3

### Matrix Tablets with Hydrophilic Excipients, Enteric Coating and a Pore-Former

| | |
|---|---|
| Ingredient | mg/Tablet |

Core Tablet

| | |
|---|---|
| Lamotrigine | 200 |
| Hydroxypropyl methylcellulose | 100 |
| (Methocel ™ K100LVCR) | |
| Lactose | 98 |
| Magnesium stearate | 2 |

Coating

| | |
|---|---|
| Methacrylic acid-ethyl acrylate | 8 |
| polymer (Eudragit L30D55) | |
| Hydroxypropyl methylcellulose | 24 |
| (Methocel ™ E5) | |
| Triethyl citrate | 8 |
| Talc | 2 |
| Water* | 95 |
| Acetone* | 45 |

*Evaporates during processing.

Manufacturing Process:

Core Tablets:

1) Lamotrigine, Methocel K100LVCR and lactose were sifted through a BSS #40 sieve and mixed together.

2) Magnesium stearate was sifted through a BSS #60 sieve and mixed with step 1 blend.

3) The blend was compressed into tablets using 14.7×6.7 mm punches.

Coating:

4) Triethyl citrate, talc and Methocel E5 were combined with a mixture of water and acetone and stirred.

5) Eudragit L30D55 was added to the step 4 dispersion and stirred at a slow speed for 45 minutes.

6) Step 3 tablets were coated with step 5 coating dispersion.

An in vitro dissolution study of the prepared tablets was conducted, using the procedure of Example 1. Results are tabulated below:

| | Cumulative % of |
|---|---|
| Hours | Drug Released |
| 1 | 3 |
| 2 | 8 |
| 4 | 21 |
| 8 | 58 |
| 12 | 91 |

### EXAMPLE 4

### Matrix Tablets with Hydrophilic Excipients and Semi-Permeable Coating

| | |
|---|---|
| Ingredient | mg/Tablet |

Core Tablet

| | |
|---|---|
| Lamotrigine | 200 |
| Hydroxypropyl methylcellulose | 100 |
| (Methocel ™ K100LVCR) | |
| Lactose | 98 |
| Magnesium stearate | 2 |

Coating

| | |
|---|---|
| Methacrylic acid-ethyl acrylate | 17 |
| (Eudragit RL100) | |
| Methacrylic acid-ethyl acrylate | 17 |
| (Eudragit RS100) | |
| Triethyl citrate | 4 |
| Talc | 2 |
| Water* | 34 |
| Acetone* | 324 |

*Evaporates during processing.

Manufacturing Procedure:

Core Tablets:

1) Lamotrigine, Methocel K100LVCR and lactose were sifted through a BSS #40 sieve and mixed together.

2) Magnesium stearate was sifted through a BSS #60 sieve and mixed with the step 1 blend.

3) The blend of 2 was compressed into tablets using 14.7×6.7 mm punches.

Coating:

4) Triethyl citrate, talc and Methocel 5 cps were combined with a mixture of water and acetone and stirred.

5) Eudragit RL100 and Eudragit RS100 were added to the step 4 dispersion and stirred at a slow speed for 45 minutes.

6) Step 3 tablets were coated with step 5 coating dispersion.

### EXAMPLES 5-8

### Matrix Tablets with Hydrophilic Excipients, Enteric Coating and a Pore-Former

| | mg/Tablet | | | |
|---|---|---|---|---|
| Ingredient | Example 5 | Example 6 | Example 7 | Example 8 |

Core Tablet

| | | | | |
|---|---|---|---|---|
| Lamotrigine | 200 | 200 | 200 | 200 |
| Hydroxypropyl | 100 | 100 | 100 | 100 |
| methylcellulose | | | | |
| (Methocel ™ | | | | |
| K100LVCR) | | | | |
| Lactose | 98 | 98 | 98 | 98 |
| Magnesium stearate | 2 | 2 | 2 | 2 |

Coating

| | | | | |
|---|---|---|---|---|
| Methacrylic acid-ethyl | 5.92 | 2.74 | 4.63 | 3.21 |
| acrylate polymer | | | | |
| (Eudragit L-100-55) | | | | |
| Hydroxypropyl | 13.81 | 10.95 | 18.53 | 9.63 |
| methylcellulose | | | | |
| (Methocel ™ E5) | | | | |
| Triethyl citrate | 5.11 | 3.51 | 5.95 | 3.33 |
| Talc | 1.16 | 0.8 | 1.36 | 0.76 |
| Acetone* | 45 | 45 | 45 | 45 |
| Water* | 95 | 95 | 95 | 95 |
| Total | 426 | 418 | 430.47 | 416.93 |

*Evaporates during processing.

Manufacturing process: similar to that described in Example 3.

In vitro dissolution testing was conducted using the procedure of Example 1, and results are below:

Cumulative % of Drug Released

| Hours | Example 5 | Example 6 | Example 7 | Example 8 |
|---|---|---|---|---|
| 1 | 3 | 6 | 3 | 6 |
| 2 | 7 | 13 | 8 | 12 |
| 4 | 17 | 33 | 21 | 29 |
| 8 | 48 | 77 | 58 | 74 |
| 12 | 82 | 101 | 91 | 101 |

### EXAMPLES 9-12

### Matrix Tablets with Hydrophilic Excipients, Enteric Coating and a Pore-Former

| | mg/Tablet | | | |
|---|---|---|---|---|
| | | Example | Example | Example |
| Ingredient | Example 9 | 10 | 11 | 12 |

Core Tablet

| | | | | |
|---|---|---|---|---|
| Lamotrigine | 200 | 200 | 200 | 200 |
| Hydroxypropyl | 45.36 | — | — | — |
| methylcellulose | | | | |
| (Methocel K4 M) | | | | |
| Hydroxypropyl | 62.64 | 100 | 100 | 100 |
| methylcellulose | | | | |
| (Methocel 100LVCR) | | | | |
| Lactose | 90.4 | 98 | 98 | 98 |
| Magnesium stearate | 1.6 | 2 | 2 | 2 |

Coating

| | | | | |
|---|---|---|---|---|
| Methacrylic acid-ethyl | 7.314 | 8.71 | 12.2 | — |
| acrylate polymer | | | | |
| (Eudragit L30D55) | | | | |
| PVP K30 | 7.314 | — | — | — |
| Methocel E5 | — | 8.71 | 5.22 | — |
| Methocel E15 | — | — | — | 36.48 |
| Triethyl citrate | 2.853 | 1.7 | 1.7 | 4.56 |
| Talc | 2.51 | 1 | 1 | 4.56 |
| Water* | 135 | 135 | 135 | 135 |
| Total | 420 | 420.12 | 420.12 | 445.6 |

*Evaporates during processing.

Manufacturing process: similar to that described in Example 2.

In vitro dissolution testing was conducted using the method of Example 1, and results are below:

Cumulative % of Drug Released

| | | Example | Example | Example |
|---|---|---|---|---|
| Hours | Example 9 | 10 | 11 | 12 |
| 1 | 7 | 9 | 5 | 7 |
| 2 | 16 | 16 | 15 | 21 |
| 4 | — | 34 | 36 | 48 |
| 8 | 50 | 64 | 73 | 83 |
| 12 | 73 | 88 | 98 | 97 |

### EXAMPLES 13-14

### Matrix Tablets with Hydrophobic Excipient, Enteric Coating and Pore-Former

mg/Tablet

| Ingredient | Example 13 | Example 14 |
|---|---|---|
| Lamotrigine | 200 | 200 |
| Stearic acid | 8 | — |
| Ethylcellulose 7 cPs | — | 12 |
| Lactose | 166 | 164 |
| PVP K30 | 20 | 20 |
| Talc | 2 | 2 |
| Colloidal silicon dioxide | 2 | — |
| Magnesium stearate | 2 | 2 |
| TOTAL | 400 | 400 |

Manufacturing Process:

1) Lamotrigine, stearic acid (for Example 13) or ethylcellulose 7 cps (for Example 14), lactose, and PVP K30 were sifted through a BSS #40 sieve and mixed together.

2) For Example 13, magnesium stearate, colloidal silicon dioxide and talc were sifted through a BSS #60 sieve and mixed with the step 1 blend.

3) For Example 14, magnesium stearate and talc were sifted through a BSS #60 sieve and mixed with the step 1 blend.

4) The mixtures of steps 2 and 3 were compressed into tablets using 14.7×6.7 mm punches.

In vitro dissolution evaluation was conducted using the procedure of Example 1, and the results are below:

Cumulative % of Drug
Released

| Hours | Example 13 | Example 14 |
|---|---|---|
| 1 | 18 | 9 |
| 2 | 25 | 16 |
| 4 | 38 | 34 |
| 8 | 61 | 64 |
| 12 | 79 | 88 |

### EXAMPLE 15

### Matrix Tablets with Hydrophobic Excipient, Enteric Coating and Pore-Former

| | |
|---|---|
| Ingredient | mg/Tablet |

Core Tablet

| | |
|---|---|
| Lamotrigine | 200 |
| Ethylcellulose 7 FP | 20 |
| Lactose | 156 |
| PVP K30 | 20 |
| Magnesium stearate | 2 |
| Talc | 2 |

Coating

| | |
|---|---|
| Methacrylic acid-ethyl acrylate | 24.38 |
| polymer (Eudragit L30D55) | |
| Povidone K30 | 7.314 |
| Triethyl citrate | 2.854 |
| Talc | 2.518 |
| Water* | 163 |

*Evaporates during processing.

Manufacturing Process:

Core Tablets:

1) Lamotrigine, ethylcellulose, lactose and PVP were shifted through a BSS #40 sieve and mixed together.

2) Magnesium stearate and talc were sifted through a BSS #60 sieve and mixed with the step 1 blend.

3) The blend of step 2 was compressed into tablets using 14.7×6.7 mm punches.

Coating:

4) Triethyl citrate, talc and povidone were combined with water and stirred.

5) Eudragit L30D55 was added to the Step 4 dispersion and stirred at a slow speed for 45 minutes.

6) Step 3 tablets were coated with step 5 coating dispersion to produce a 5% weight gain.

### EXAMPLE 16

### Tablets with Surface-Modified Lamotrigine

| | |
|---|---|
| Ingredient | mg/Tablet |

Core Tablet

| | |
|---|---|
| Lamotrigine | 200 |
| Stearic acid | 8 |
| Lactose | 166 |
| Povidone K30 | 20 |
| Magnesium stearate | 2 |
| Talc | 2 |
| Collidal silicon dioxide | 2 |

Coating

| | |
|---|---|
| Methacrylic acid-ethyl acrylate | 24.38 |
| polymer (Eudragit L30D55) | |
| PVP K30 | 7.314 |
| Triethyl citrate | 2.854 |
| Talc | 2.518 |
| Water* | 163 |

*Evaporates during processing.

Manufacturing Process:

Core Tablets:

1) Stearic acid was melted at 60° C.

2) To the step 1) melt, lamotrigine was added and mixed thoroughly, then cooled to room temperature to get a solid mass.

3) Step 2) mass was sifted through a BSS #40 sieve to get granules.

4) Lactose and povidone were sifted through a BSS #40 sieve and mixed with step 3) granules.

5) Magnesium stearate, talc and colloidal silicon dioxide were sifted through a BSS #60 sieve and mixed with step 4) granules.

6) Step 5) blend was compressed into tablets using 14.7×6.7 mm punches.

Coating:

7) Triethyl citrate, talc and PVP were combined with water and stirred.

8) Eudragit L30D55 was added to the step 7) dispersion and stirred at a slow speed for 45 minutes.

9) Step 6) tablets were coated with step 8) coating dispersion to produce a 5% weight gain.

### EXAMPLE 17

### Lamotrigine Tablets with Eudragit Matrix

| Ingredient | mg/Tablet |
|---|---|
| Lamotrigine | 200 |
| Lactose monohydrate | 158 |
| Methacrylic acid-ethyl acrylate | 20 |
| polymer (Eudragit L30D55) | |
| Methacrylic acid-ethyl acrylate | 20 |
| polymer (Eudragit RL 30D) | |
| Magnesium stearate | 2 |

Manufacturing Process:

1) Lactose monohydrate and lamotrigine were sifted through a BSS #40 sieve and mixed.

2) Step 1) blend was granulated with a mixture of Eudragit L 30D 55 and Eudragit RL 30 D dispersion.

3) Step 2) granules were dried at 60° C. and sifted through a BSS #40 sieve.

4) Magnesium stearate was sifted through a BSS #60 sieve and mixed with step 3) granules.

5) Step 4) blend was compressed into tablets using 14.7×6.7 mm punches.

### EXAMPLE 18

### Immediate-Release Lamotrigine Tablets with Semi-Permeable Coating

| | |
|---|---|
| Ingredient | mg/Tablet |

Core Tablet

| | |
|---|---|
| Lamotrigine | 200 |
| Lactose | 198 |
| Magnesium stearate | 2 |

Coating

| | |
|---|---|
| Cellulose acetate | 10 |
| Hydroxypropyl methylcellulose | 4.9 |
| (Methocel E5) | |
| PEG 4000 | 0.2 |
| Acetone* | 342 |
| Water* | 38 |

*Evaporates during processing.

Manufacturing Process:

Core Tablets:

1) Lamotrigine and lactose were sifted through a BSS #40 sieve and mixed.

2) Magnesium stearate was sifted through a BSS #60 sieve and mixed with the step 1) blend.

3) Step 2) blend was compressed into tablets using 14.7×6.7 mm punches.

Coating:

4) Dissolved PEG and Methocel E5 in water.

5) Step 4) solution was added to acetone while stirring.

6) Cellulose acetate was added to the step 5) solution while stirring to form a clear solution.

7) Step 3) tablets were coated with step 6) coating solution to produce a 5% weight gain.

### EXAMPLE 19

### Immediate-Release Lamotrigine Tablets with Extended-Release Coating

| | |
|---|---|
| Ingredient | mg/Tablet |

Core Tablet

| | |
|---|---|
| Lamotrigine | 200 |
| Lactose | 198 |
| Magnesium stearate | 2 |

Coating

| | |
|---|---|
| Ethylcellulose 7 cP | 10 |
| Hydroxypropyl methylcellulose | 9.8 |
| (Methocel E5) | |
| Triethyl citrate | 0.2 |
| Isopropyl alcohol* | 342 |
| Water* | 38 |

*Evaporates during processing.

Manufacturing Process:

Core Tablets:

1) Lamotrigine and lactose were sifted through a BSS #40 sieve and mixed.

2) Magnesium stearate was sifted through a BSS #60 sieve and mixed with the step 1) blend.

3) Step 2) blend was compressed into tablets using 14.7×6.7 mm punches.

Coating:

4) Dissolved Methocel E5 in water.

5) Dissolved ethylcellulose and triethyl citrate in isopropyl alcohol.

6) Step 4) and 5) solutions were mixed together to get a homogenous solution.

7) Step 3) tablets were coated with step 6) coating solution to produce a 5% weight gain.

### EXAMPLE 20

### Delayed-Release and Extended-Release Pellets

| | |
|---|---|
| Ingredient | mg/Unit Dose |

Drug-Loaded Pellets

| | |
|---|---|
| Sugar spheres (30/35 mesh) | 50 |
| Lamotrigine (micronised) | 200 |
| Hydroxypropyl methylcellulose | 20 |
| (Methocel E5) | |
| Water* | 1980 |
| Wt. of pellets | 270 |

Extended-Release Pellets

| | |
|---|---|
| Drug-loaded pellets | 135 |
| Ethylcellulose 7 cP | 6.5 |
| Hydroxypropyl methylcellulose | 6.5 |
| (Methocel E5) | |
| Triethyl citrate | 0.5 |
| Isopropyl alcohol* | 230.85 |
| Water* | 25.65 |
| Total weight | 148.5 |

Delayed-Release Pellets

| | |
|---|---|
| Drug-loaded pellets | 135 |
| Methacrylic acid-ethylacrylate | 6.25 |
| polymer (Eudragit L 100-55) | |
| Triethyl citrate | 0.5 |
| Isopropyl alcohol* | 230.85 |
| Water* | 25.65 |
| Total weight | 141.75 |

*Evaporates during processing.

Manufacturing Process:

Drug-Loaded Pellets:

1) Methocel E5 was dissolved in water.

2) Lamotrigine was added to the step 1) solution with stirring.

3) Sugar spheres were coated with the step 2) suspension using a fluidized bed coater.

Extended-Release Pellets:

4) Ethylcellulose, Methocel E5 and triethyl citrate were dissolved in a mixture of isopropyl alcohol and water.

5) Drug-loaded pellets of step 3) were coated with step 4) solution to produce a 10% weight gain.

Delayed-Release Pellets:

6) Eudragit L 100-55 and triethyl citrate were dissolved in a mixture of isopropyl alcohol and water.

7) Drug-loaded pellets of step 3) were coated with step 6) solution to produce a 5% weight gain.

Pellet Mixing:

8) Equal weights of step 5) and step 7) pellets were mixed.

Step 8) pellets can be filled into capsules, or compressed into tablets (optionally, together with additional pharmaceutical excipients).

We claim:

1. A pharmaceutical formulation comprising lamotrigine or a salt thereof, wherein the formulation comprises a core having a coating comprising a polymer that controls release of lamotrigine into an aqueous environment, the coating optionally having pores or channels.

2. The pharmaceutical formulation of claim 1, wherein a polymer comprises a copolymer of methacrylic acid and ethyl acrylate.

3. The pharmaceutical formulation of claim 1, wherein pores or channels are formed in a coating by an included water-soluble pore-former, upon exposure to an aqueous environment.

4. The pharmaceutical formulation of claim 3, wherein a water-soluble pore-former comprises a polymer.

5. The pharmaceutical formulation of claim 1, wherein the core is a tablet.

6. The pharmaceutical formulation of claim 1, wherein the core provides immediate release or modified release of contained lamotrigine.

7. The pharmaceutical formulation of claim 1, wherein the core provides modified release of contained lamotrigine and comprises at least one of hydrophobic excipients and lamotrigine surface-modifying excipients.

8. The pharmaceutical formulation of claim 1, wherein a coating comprises a polymer having pH-independent aqueous solubility.

9. The pharmaceutical formulation of claim 1, wherein a coating comprises a polymer having pH-dependent aqueous solubility.

10. The pharmaceutical formulation of claim 1, wherein a coating comprises a combination of polymers having pH-dependent aqueous solubility and pH-independent aqueous solubility.

11. The pharmaceutical formulation of claim 1, comprising two or more portions of cores having different coatings.

12. The pharmaceutical formulation of claim 12, wherein a portion of cores provides extended release of lamotrigine.

13. The pharmaceutical formulation of claim 12, wherein a portion of cores provides delayed release of lamotrigine.

14. A pharmaceutical formulation comprising a tablet containing lamotrigine or a salt thereof and having a coating comprising a methacrylic acid-ethyl acrylate copolymer.

15. The pharmaceutical formulation of claim 14, wherein the tablet comprises a hydrophilic polymer.

16. The pharmaceutical formulation of claim 14, wherein pores or channels are formed in a coating by an included water-soluble pore-former, upon exposure to an aqueous environment.

17. The pharmaceutical formulation of claim 16, wherein a water-soluble pore-former comprises a polymer.

18. A pharmaceutical formulation comprising pharmacologically inert core particles coated with a composition comprising lamotrigine or a salt thereof and a hydrophilic polymer, the coated particles being further coated with a composition comprising a methacrylic acid-ethyl acrylate copolymer.

19. The pharmaceutical formulation of claim 18, wherein pores or channels are formed in a methacrylic acid-ethyl acrylate copolymer coating by an included water-soluble pore-former, upon exposure to an aqueous environment.

20. The pharmaceutical formulation of claim 16, wherein a water-soluble pore-former comprises a polymer.
